(19) United States
(12) Patent Application Publication
(10) Pub. No.: US 2018/0325427 A1
(43) Pub. Date: Nov. 15, 2018

(54) IMAGING DEVICE, BIOMETRIC AUTHENTICATION DEVICE, AND SEMICONDUCTOR LASER

(71) Applicant: SHARP KABUSHIKI KAISHA, Sakai City, Osaka (JP)

(72) Inventors: SHIN ITOH, Sakai City (JP); KAZUHIRO TSUCHIDA, Sakai City (JP); TOHRU MURATA, Sakai City (JP); HIROAKI YAMAMOTO, Sakai City (JP); AKIRA ARIYOSHI, Sakai City (JP); KEISUKE MIYAZAKI, Sakai City (JP); YOSHIKI SOTA, Sakai City (JP)

(21) Appl. No.: 15/775,902

(22) PCT Filed: Sep. 29, 2016
(86) PCT No.: PCT/JP2016/078860

§ 371 Date: May 14, 2018

(30) Foreign Application Priority Data
Nov. 16, 2015 (JP) 2015-224155

(51) Int. Cl. A61B 5/1171; G06T 1/00; H01S 5/022; G06K 9/00

(52) U.S. Cl. CPC A61B 5/1171; G06K 9/00013; H01S 5/022; G06T 1/0007

(57) ABSTRACT

Provided is an imaging device capable of improving imaging accuracy while suppressing increase in a size of the device and cost thereof. An imaging device includes: a light source which is a semiconductor laser that radiates infrared light to an imaging target; a polarizing filter that blocks light that is reflected by a surface of the imaging target and transmits light that is reflected inside the imaging target; and an image sensor that receives the light that is transmitted through the polarizing filter and captures an image of the imaging target.

### TECHNICAL FIELD

The present invention relates to an imaging device that captures an image of a site including biological information, a biometric authentication device that includes the imaging device, and a semiconductor laser for the imaging device.

### BACKGROUND ART

Recently, a biometric authentication technique has been developed in which a user is authenticated by using biological information such as a vein pattern of a palm or a finger, a finger print, or a palm print. A biometric authentication device using such a biometric authentication technique uses, as a method of acquiring the biological information, a method in which an image of a living body is captured with use of light such as transmission light or reflection light to thereby acquire the biological information.

In a case where a living body such as a vein, which is positioned under a skin, is photographed, diffusion light which is reflection light that is diffused inside a palm or a finger and returns therefrom is used. In this case, surface reflection light that is reflected by a skin becomes noise, and therefore it is difficult to authenticate the living body when the surface reflection light and the diffusion light are superposed.

Then, a method of removing the surface reflection light that is reflected by a skin is proposed in PTL 1. Specifically, PTL 1 discloses a complexion measuring device which detects reflection light by causing each of incident light from a lamp light source and the reflection light thereof to pass through a polarizer, and thereby removes surface reflection light that is reflected by a skin surface,

### CITATION LIST

### Patent Literature

PTL 1: Japanese Unexamined Patent Application Publication No. 2002-200050 (published on Jul. 16, 2002)

### SUMMARY OF INVENTION

### Technical Problem

However, in a case where a lamp light source as disclosed in PTL 1 is used as a light source, a wavelength filter is required to be used in order to radiate light having a specific wavelength, and furthermore, a polarizing filter is required to be used in order to radiate light having a specific polarization plane.

In a case where filters such as the wavelength filter and the polarizing filter are used, intensity of light that is transmitted through the filters is reduced to be half or less. This makes imaging accuracy deteriorated, and, as a result, the imaging accuracy is lowered. Further, a size of the light source is increased by providing the filters, and thus the light source is unsuitable to be mounted on mobile equipment. There is also a problem that cost of the light source becomes high due to high cost of the filters.

The invention is made in view of the aforementioned problems and an object thereof is to provide an imaging device capable of improving imaging accuracy while suppressing increase in a size of the device and cost thereof, a biometric authentication device, and a semiconductor laser.

### Solution to Problem

In order to solve the aforementioned problems, an imaging device according to an aspect of the invention is an imaging device that captures an image of a site including biological information as an imaging target and includes: a semiconductor laser that radiates infrared light to the imaging target; a polarizing filter that blocks light that is reflected by a surface of the imaging target and transmits light that is reflected inside the imaging target; and an image sensor that receives the light that is transmitted through the polarizing filter and captures an image of the imaging target.

Furthermore, in order to solve the aforementioned problems, a semiconductor laser for an imaging device that captures am image of a site including biological information, according to an aspect of the invention, radiates infrared light, has a polarization ratio which is not less than 3, and is an eye-safe laser.

### Advantageous Effects of Invention

According to an aspect of the present invention, it is possible to improve imaging accuracy while suppressing increase in a size of the device and cost thereof.

### BRIEF DESCRIPTION OF DRAWINGS

FIG. 1 is a schematic view illustrating an imaging device according to an embodiment of the invention.

FIG. 2 is a view illustrating a polarization state of light radiated from a light source according to the embodiment of the invention, in which (a) illustrates a polarization state when the light is reflected by a surface of an imaging target, and (b) illustrates a polarization state when the light is reflected inside the imaging target.

FIG. 3 is an external view illustrating the light source according to an embodiment of the invention.

FIG. 4 illustrates diffusion of light in a diffusion member of the light source according to the embodiment of the invention.

FIG. 5 illustrates a shape of a surface of the diffusion member of the light source according to the embodiment of the invention.

FIG. 6 is a view illustrating a polarization direction of light radiated from a semiconductor laser chip according to an embodiment of the invention, in which (a) illustrates the polarization direction when the semiconductor laser chip is horizontally placed, and (b) illustrates the polarization direction when the semiconductor laser chip is vertically placed.

FIG. 7 is a view illustrating a polarization state of the light radiated from the semiconductor laser chip according to the embodiment of the invention, in which (a) illustrates the polarization state when the semiconductor laser chip is horizontally placed, and (b) illustrates the polarization state when the semiconductor laser chip is vertically placed.

FIG. 8(a) is a top view illustrating portable electronic equipment that includes an imaging device according to an embodiment of the invention, and (b) is a view illustrating a state in which light is made vertically incident on an imaging target from a light source.

### DESCRIPTION OF EMBODIMENTS

### Embodiment 1

### (Configuration of Imaging Device)

An imaging device according to the invention is an imaging device that captures an image of a living body of a user for performing biometric authentication. Specifically, an image of a living body of a user is captured by using reflection light of light radiated to the living body, which is diffused inside the living body and returns therefrom. The living body as an imaging target is a site including biological information, and examples thereof include a finger and a palm. When an image of the site including biological information is captured by the imaging device, the biological information such as a vein pattern of a finger or a palm, a finger print, or a palm print is able to be acquired.

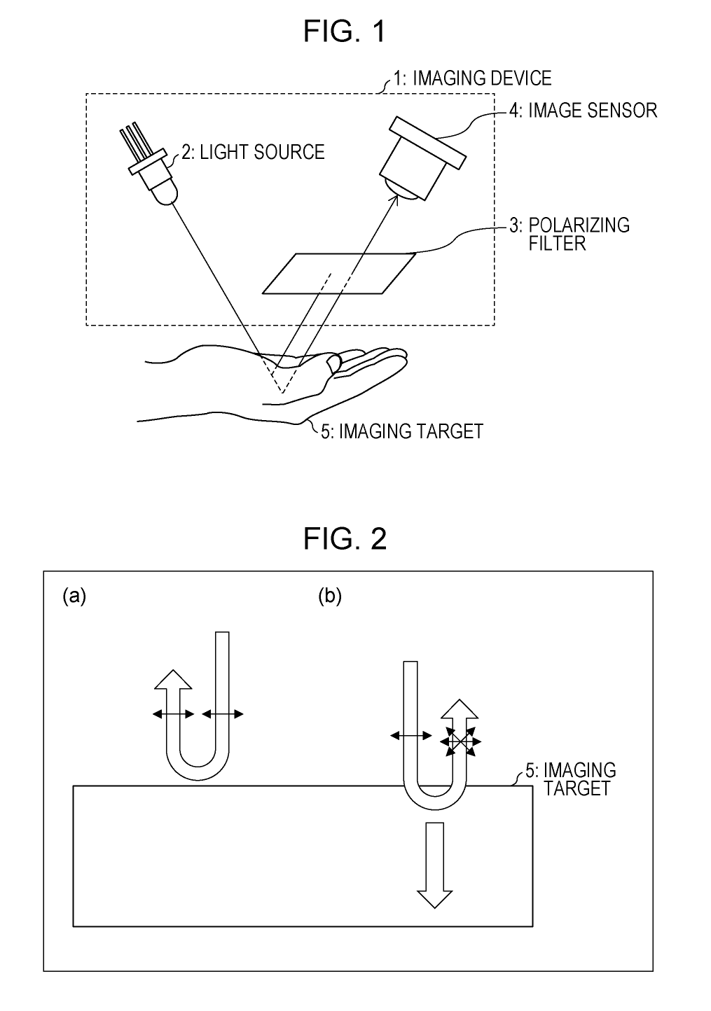

Hereinafter, a case where a vein pattern of a palm of a user is acquired by capturing an image of the palm, will be described as an example, and the imaging device according to an embodiment of the invention is described with reference to FIG. 1. FIG. 1 is a schematic view illustrating an imaging device 1 according to the present embodiment.

The imaging device 1 includes a light source 2, a polarizing filter 3, and an image sensor 4 as illustrated in FIG. 1. Note that, for ease of understanding component members of the imaging device 1, FIG. 1 illustrates only a schematic configuration.

The light source 2 is a semiconductor laser which radiates infrared light and irradiates an imaging target 5 with the light so that biological information has sufficient contrast on an image captured by the image sensor 4. In the semiconductor laser, light radiated from an active layer of a semiconductor laser chip repeatedly reciprocates between two cleaved end surfaces of the semiconductor laser chip and then is emitted from one of the end surfaces, and therefore light emitted from the semiconductor laser is light having a specific polarization plane.

Note that, in FIG. 1, the light source 2 is arranged so that light radiated from the light source 2 is obliquely incident on the imaging target 5, but the invention is not necessarily limited thereto. For example, the light source 2 may be arranged so that the light radiated from the light source 2 is vertically incident on the imaging target 5.

Though details will be described later, a part of the light, which is radiated from the light source 2 to the imaging target 5, enters the imaging target 5, and, while repeatedly being diffused, a part of the light is absorbed by a vein in this process and is then finally emitted from a skin surface. Furthermore, a part of the light radiated from the light source 2 to the imaging target 5 is mirror-reflected by a surface of the imaging target 5.

The polarizing filter 3 is arranged between the image sensor 4 and the imaging target 5 and is a filter which blocks a part of the light radiated from the light source 2 to the imaging target 5, which is mirror-reflected by the surface of the imaging target 5, and transmits a part of the light, which is diffuse-reflected inside the imaging target 5. Specifically, the polarizing filter 3 is arranged so that a light-transmitting surface thereof is orthogonal to a main polarization plane of the light source 2. Accordingly, though details will be described later, the polarizing filter 3 blocks a part of the light radiated from the light source 2, which is mirror-reflected by the surface of the imaging target 5, and transmits a part of the light, which is diffuse-reflected inside the imaging target 5.

The image sensor 4 receives the light that is transmitted through the polarizing filter 3 and captures an image of the imaging target 5. Specifically, the image sensor 4 receives the light that is transmitted through the polarizing filter 3, that is, the light that is diffuse-reflected inside the imaging target 5, and thereby generates a captured image that has brightness distribution according to the biological information.

### (Method of Capturing Image of Living Body)

In the imaging device 1 according to the present embodiment, as a method of capturing an image of the imaging target 5, a method is adopted in which an image of a living body of a user is captured by using reflection light of light radiated to the living body, which is diffused inside the living body and returns therefrom. Hereinafter, a case where a vein pattern of a palm of a user is acquired by capturing an image of the palm will be described as an example, and the imaging method by the imaging device 1 is described with reference to FIG. 2. FIG. 2 is a view illustrating a polarization state of light radiated from the light source 2, in which (a) illustrates a polarization state when the light is reflected by the surface of the imaging target 5, and (b) illustrates a polarization state when the light is reflected inside the imaging target 5.

When light is radiated from, the light source 2 to the imaging target 5, a part of the radiated light is mirror-reflected by the surface of the imaging target 5 as illustrated in FIG. 2(a). Specifically, a part of the light radiated from the light source 2 is reflected at a reflection angle equivalent to an incident angle with respect to a line normal to the surface of the imaging target 5. The light that is mirror-reflected by the surface of the imaging target 5 is reflected in a state where a polarization plane of incident light is maintained.

On the other hand, a different part of the light radiated from the light source 2 is diffuse-reflected inside the imaging target 5 as illustrated in FIG. 2(b). Specifically, the different part of the light radiated from the light source 2 enters the imaging target 5 and, while repeatedly being diffused, is emitted from a skin surface. In this process, a part of the light is absorbed by a vein. The light that is diffuse-reflected inside the imaging target 5 is diffused in various directions and is therefore reflected in a state where no specific polarization plane is included.

The light mirror-reflected by the surface of the imaging target 5 and the light diffuse-reflected inside the imaging target 5 reach the polarizing filter 3. The light-transmitting surface of the polarizing filter 3 is arranged so as to be orthogonal to the main polarization plane of the light radiated from the light source 2, and thus the light mirror-reflected by the surface of the imaging target 5 is not transmitted through the polarizing filter 3. On the other hand, a part of the light that is diffuse-reflected inside the imaging target 5, which has a polarization plane that is the same as the light-transmitting surface of the polarizing filter 3, is only transmitted through the polarizing filter 3.

In this manner, a part of the light diffuse-reflected inside the imaging target 5 is transmitted through the polarizing filter 3 and is received by the image sensor 4. Since intensity of light diffuse-reflected in a vein part of the inside of the imaging target 5 is lower than intensity of light diffuse-reflected in a part other than the vein, an image in which brightness distribution according to the vein that is, according to biological information is reproduced is generated by the image sensor 4.

When both the diffuse-reflected light and the mirror-reflected light are incident on the image sensor 4, on the captured image, unevenness of brightness occurs between an area where the mirror-reflected light reaches and an area where the mirror-reflected light does not reach, and there is a possibility that the brightness distribution of the diffuse-reflected light according to the biological information is not reproduced on the captured image. However, the polarizing filter 3 blocks the light mirror-reflected by the surface of the imaging target 5, and accordingly the image in which the brightness distribution of the diffuse-reflected light according to the biological information is excellently reproduced is able to be captured.

Since the light source 2 is composed of a semiconductor laser in the present embodiment, light radiated from the light source 2 has a specific wavelength and a specific polarization plane. Thus, it is not necessary to use a filter such as a wavelength filter or a polarizing filter for the light source 2.

Accordingly, it is possible to suppress loss of light, that is, reduction of intensity of light due to transmission through a filter. Thus, imaging accuracy of the imaging device 1 according to the present embodiment is improved. In addition, since the filter is not required, the light source 2 is able to be made compact and also able to be mounted on mobile equipment. Furthermore, since the filter is not required to be provided, it is possible to suppress cost of the light source 2 so as to be low.

In a biometric authentication device including the imaging device 1 according to the present embodiment, since the imaging accuracy of the imaging device 1 is improved, biometric authentication accuracy of the biometric authentication device is also improved. In this manner, the biometric authentication device including the imaging device 1 according to the present embodiment is also included in the scope of the invention.

### [Application Example of Embodiment 1]

In Embodiment 1 described above, in order to effectively extract biological information in a living body, which is represented by a vein pattern, by arranging the polarizing filter 3 so that the light-transmitting surface thereof is orthogonal to the main polarization plane of the light source 2, information of a surface (for example, a finger print, a palm print, a wound, a wrinkle, or the like) of the imaging target 5 is excluded, and information of the inside (for example, a vein pattern or the like) is extracted.

However, the invention is not necessarily limited thereto. For example, when the polarizing filter 3 is arranged so that the light-transmitting surface is parallel to the main polarization plane of the light source 2, it is possible to exclude information inside the imaging target 5 and more effectively collect information of the surface, such as a finger print, a palm print, a wound, or a wrinkle, which is described above.

In this case, when S-polarized light is made obliquely incident on the imaging target 5 from the light source 2 and the polarizing filer 3 is arranged so that the light-transmitting surface is parallel to a polarization plane of the S-polarized light, the information of the surface of the imaging target 5 is able to be effectively collected. Note that, a method of radiating S-polarized light or P-polarized light from the light source 2 will be described in detail in Embodiment 3.

### Embodiment 2

In general, when a semiconductor laser is used in a living space, a living body of a user, such as a retina, may be damaged. Therefore, the light source 2 that is used in the imaging device 1 according to the present embodiment is composed of an eye-safe laser in order to secure safety of a living body of a user, specially, safety of an eye.

### (Configuration of Light Source)

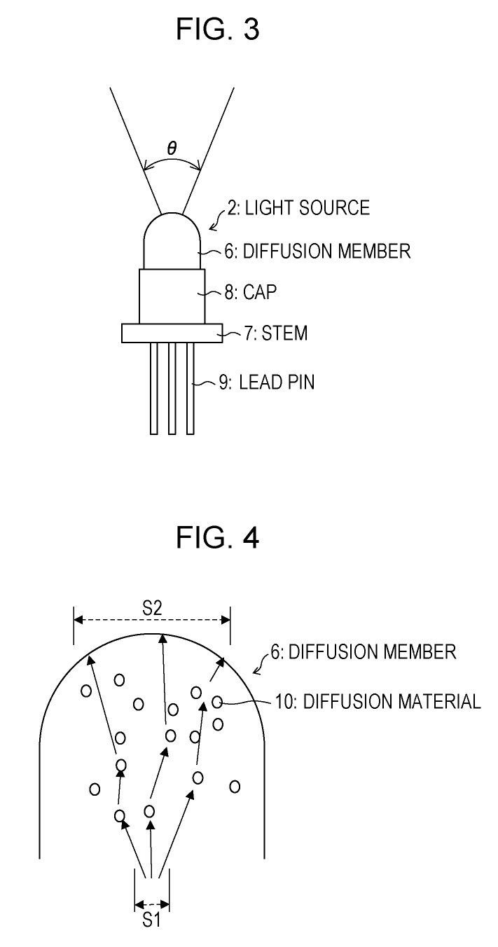

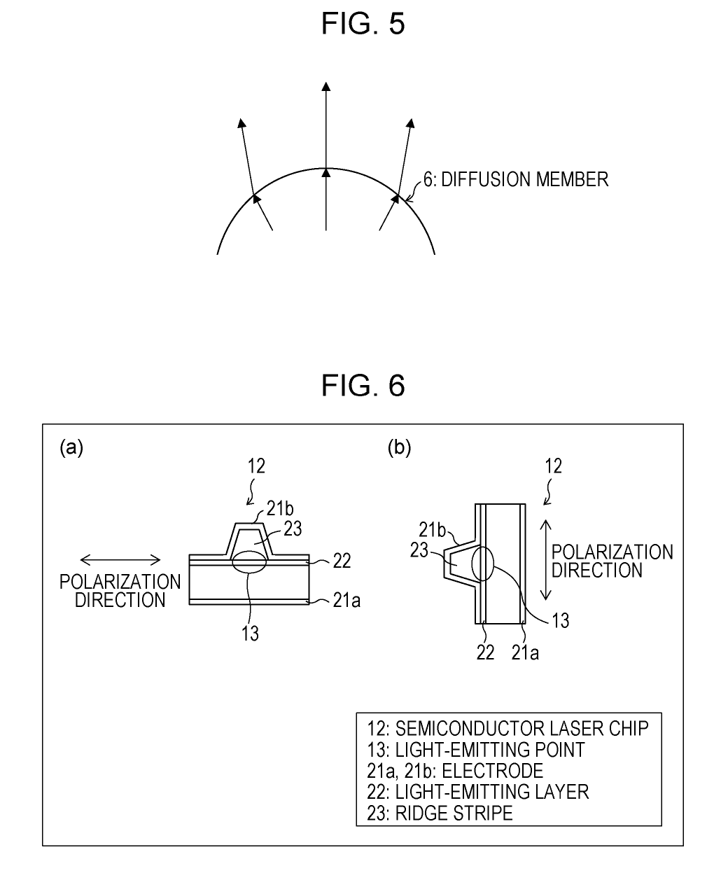

The light source 2 according to the present embodiment will be described with reference to FIG. 3 to FIG. 5. FIG. 3 is an external view illustrating the light source 2, FIG. 4 illustrates diffusion of light in a diffusion member 6 of the light source 2, and FIG. 5 illustrates a shape of a surface of the diffusion member 6 of the light source 2.

As illustrated in FIG. 3, the light source 2 includes a semiconductor laser chip (not illustrated), the diffusion member 6, a stem 7, a cap 8, and lead pins 9. The stem 7 is a portion serving as a base and the cap 8 is fixed on one end surface thereof. The stem 7 has a plurality of through holes for arranging the lead pins 9 and each of the lead pins 9 is fixed to the stem 7 in a state of being inserted into a corresponding one of the through holes.

The cap 8 is an exterior member that stores various components including the semiconductor laser chip. The cap 8 includes, on an end on a side opposite to the stem 7, the diffusion member 6 that transmits light emitted from the semiconductor laser chip.

As illustrated in FIG. 4, the diffusion member 6 is formed of transparent resin, glass, or the like and an inside thereof is filled with a diffusion material 10 such as filler. The filler used as the diffusion material 10 is, for example, an inorganic material such as silica, alumina, titanium oxide, or zirconia, or a compound thereof.

Light radiated from the semiconductor laser chip is diffused by the diffusion material 10 in the diffusion member 6 and emitted to an outside. Thus, although a light source size of the light source 2 is S1, the light radiated from the semiconductor laser chip is diffused in the diffusion member 6, and thus an apparent light source size of the light source 2 expands up to a size of S2. The apparent light source size of the light source 2 is enlarged in this manner to thereby secure safety of a living body of a user.

Note that, as illustrated in FIG. 5, the shape of the surface of the diffusion member 6 is a lens shape. By appropriately changing the lens shape of the surface of the diffusion member 6, a radiation angle θ of light that is radiated from the semiconductor laser chip and diffused in the diffusion member 6 is able to be controlled.

Though a method by which the eye-safe light source 2 is realized by transmitting laser light through the diffusion member 6 is described above as an example, a method of realizing the eye-safe light source 2 is not limited thereto. For example, the eye-safe light source 2 may be realized by forming a reflection surface, which is configured by an inclined surface or a carved surface, with use of a diffusion member including a diffusion material with a high concentration and radiating laser light thereto. In this case, by appropriately changing a shape of the reflection surface, the radiation angle θ of the light radiated from the semiconductor laser chip is able to be controlled.

Alternatively, a method obtained by appropriately combining the above-described method of transmitting the laser light through the diffusion member 6 and the method of reflecting the laser light by the diffusion member may be used.

### (Polarization Ratio of Light Source)

A polarization ratio is a ratio of intensity of light having the main polarization plane of the light source 2 to intensity of light having a polarization plane other than the main polarization plane of the light source 2. The polarization ratio of the light source 2 according to the present embodiment is preferably not less than 3, more preferably not less than 9.

When the polarization ratio of the light source 2 is not less than 3, that is, a part of light radiated from the light source 2, which is polarized to the main polarization plane, is not less than 75%, an image suitable for performing biometric authentication is able to be captured. When the polarization ratio of the light source 2 is not less than 9, that is, the part of the light radiated from the light source 2, which is polarized to the main polarization plane, is not less than 90%, contrast of biological information (for example, contrast between a vein part and a part other than the vein part) is clear, and therefore it is possible to capture an extremely excellent image in which brightness distribution according to the biological information is clearly reproduced.

When the polarization ratio of the light source 2 is less than 2 (that is, the part of the light radiated from the light source 2, which is polarized to the main polarization plane, is less than 67%), an amount of light of the light radiated from the light source 2, which is mirror-reflected by the surface of the imaging target 5 and transmitted through the polarizing filter 3, increases, and thus the captured image is unclear.

The polarization ratio of the light source 2 is decided in accordance with a material and a concentration of the diffusion material 10 in the diffusion member 6, and a thickness of the diffusion member 6, and a finish condition of the surface thereof. Thus, the polarization ratio of the light source 2 is able to be controlled by appropriately adjusting the concentration of the material and the thickness of the diffusion member 6 in accordance with the material that is used as the diffusion material 10.

### Embodiment 3

Regarding linearly polarized light, linearly polarized light having a plane-of-vibration of the polarized light, which is parallel to an incident plane (a plane formed by incident light and reflection light that the incident light is regularly reflected), is P-polarized light, and linearly polarized light having a plane-of-vibration of the polarized light, which is vertical to the incident plane, is S-polarized light. In general, when being obliquely incident on a target, reflectivity of the S-polarized light is higher than that of the P-polarized light. Thus, in the present embodiment, in order to reduce mirror reflectivity at the surface of the imaging target 5, the light radiated from the light source 2 is set as the P-polarized light and the P-polarized light is made obliquely incident on the imaging target 5.

When the P-polarized light is obliquely radiated from the light source 2 to the imaging target 5, it is possible to reduce light mirror-reflected by the surface of the imaging target 5. As a result, a rate of light that enters the imaging target 5 and is diffuse-reflected inside the imaging target 5 increases, and therefore imaging accuracy is able to be improved.

On the other hand, when an image of the surface of the imaging target 5 is captured, in order to reduce diffuse reflectivity inside the imaging target 5, it is desired that the light radiated from the light source 2 is set as the S-polarized light and the S-polarized light is made obliquely incident on the imaging target 5.

### (Arrangement of Semiconductor Laser Chip)

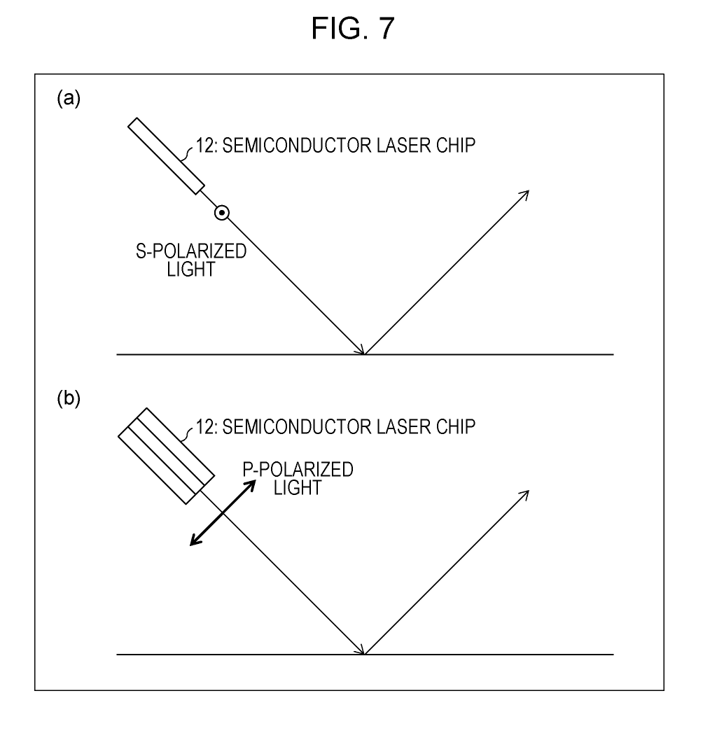

As described above, since light having a specific polarization plane is emitted from the cleaved end surfaces of the semiconductor laser chip of the light source 2, by changing a way of arranging the semiconductor laser chip, it is possible to set incident light on the imaging target 5 as the P-polarized light. This will be described with reference to FIG. 6 and FIG. 7.

FIG. 6 is a view illustrating a polarization direction of light radiated from a semiconductor laser chip 12, in which (a) illustrates the polarization direction when the semiconductor laser chip 12 is horizontally placed, and (b) illustrates the polarization direction when the semiconductor laser chip 12 is vertically placed. In addition, FIG. 7 is a view illustrating the polarization direction of the light radiated from the semiconductor laser chip, in which (a) illustrates the polarization direction when the semiconductor laser chip 12 is horizontally placed, and (b) illustrates the polarization direction when the semiconductor laser chip 12 is vertically placed.

FIG. 6 illustrates the semiconductor laser chip 12 of a ridge stripe type. Between an electrode 21a and an electrode 21b, a light-emitting layer (active layer) 22 and a ridge stripe 23 are laid. As illustrated in FIG. 6(a), when the semiconductor laser chip 12 is horizontally placed (that is, arranged so that a main surface of the semiconductor laser chip 12 is vertical to an incident plane), light emitted from a light-emitting point 13 of the semiconductor laser chip 12 is to have a polarization direction (polarization plane) vertical to the incident plane. Note that, the incident plane is a plane that is formed by light emitted from a semiconductor laser chip and reflection light that the emitted light is regularly reflected.

When light radiated from semiconductor laser chip 12 that is horizontally placed is made obliquely incident on the imaging target 5, as illustrated in FIG. 7(a), the light radiated from the semiconductor laser chip 12 becomes linearly polarized light that has a plane-of-vibration of the polarized light, which is vertical to the incident plane, that is, the S-polarized light. Accordingly, when the light radiated from the semiconductor laser chip 12 that is horizontally placed is made obliquely incident on the imaging target 5, the S-polarized light is to be incident on the imaging target 5.

On the other hand, as illustrated in FIG. 6(b), when the semiconductor laser chip 12 is vertically placed (that is, arranged so that the main surface of the semiconductor laser chip 12 is parallel to the incident plane), light emitted from the light-emitting point 13 of the semiconductor laser chip 12 is to have a polarization direction (polarization plane) parallel to the incident plane.

When light radiated from semiconductor laser chip 12 that is vertically placed is made obliquely incident on the imaging target 5, as illustrated in FIG. 7(b), the light radiated from the semiconductor laser chip 12 becomes linearly polarized light that has a plane-of-vibration of the polarized light, which is parallel to the incident plane that is, the P-polarized light. Accordingly, when the light radiated from the semiconductor laser chip 12 that is vertically placed is made obliquely incident on the imaging target 5, the P-polarized light is to be incident on the imaging target 5.

Therefore, in order to set the light radiated from the light source 2 as the P-polarized light and make the P-polarized light obliquely incident on the imaging target 5, the light source 2 may be arranged so that the semiconductor laser chip 12 is vertically placed, that is, the main surface of the semiconductor laser chip 12 is parallel to the incident plane.

In the above description, the semiconductor laser chip 12 of TE polarized light has been described as an example, but a semiconductor laser chip of TM polarized light may be used. Since all polarization planes indicated in FIG. 6 and FIG. 7 are turned by 90° in the semiconductor laser chip of the TM polarized light, exactly similar theory taking the turn into consideration is applied when the semiconductor laser chip of TM polarized light is used.

### [Additional Note]

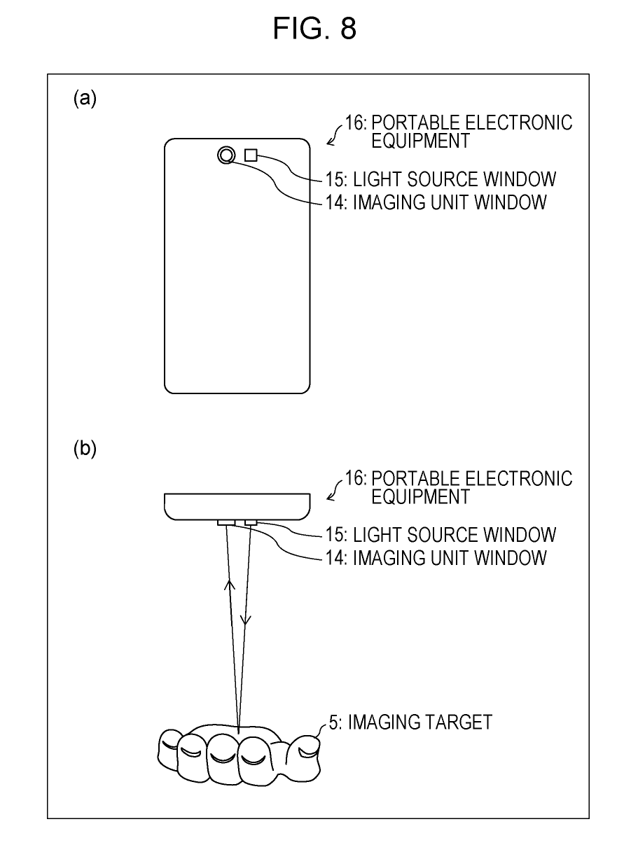

The arrangement such that the light-transmitting surface of the polarizing filter 3 is orthogonal to the main polarization plane of the light source 2 most effectively functions when light radiated from the light source 2, which is vertically incident on the imaging target 5 and vertically reflected, is detected by the image sensor 4. This will be described with reference to FIG. 8. FIG. 8(a) is a top view illustrating portable electronic equipment that includes the imaging device according to one of the embodiments of the invention, and (b) is a view illustrating a state in which, from a light source to an imaging target, light is made vertically incident and vertically reflected.

A mode in which the light radiated from the light source 2 is made vertically incident and vertically reflected with respect to the imaging target 5 is preferable, for example, in a case where the light source 2 and the image sensor 4 are arranged close to each other. Examples of the case where the light source 2 and the image sensor 4 are arranged close to each other include a case where the imaging device 1 is mounted on portable electronic equipment 16 as illustrated in FIG. 8, or the like.

The portable electronic equipment 16 illustrated in FIG. 8(a) includes a light source window 15 through which light from the light source 2 passes and an imaging unit window 14 through which light that is reflected by the imaging target 5 passes. As illustrated in FIG. 8(b), after the light radiated from the light source 2 passes through the light source window 15 and is vertically incident on the imaging target 5, light that is vertically reflected by the imaging target 5 passes through the imaging unit window 14, and enters the image sensor 4.

Whereas, when the light radiated from the light source 2 is made obliquely incident on the imaging target 5 as in the case of FIG. 1, reflectivity at a boundary of substances is deferent between an S-wave and a P-wave, and thus a polarization plane rotates around an optical axis of the light source 2 before and after reflection. As a special state in which the polarization plane does not rotate, cases of the P-polarized light and the S-polarized light are cited.

For the two kinds of polarized light, the polarization plane does not rotate before or after reflection of the light. In this case, by arranging the polarizing filter 3 so that the light-transmitting surface thereof is orthogonal to the main polarization plane of the light source 2, which is composed of the P-polarized light or the S-polarized light, it is possible to effectively remove mirror reflected light at the surface of the imaging target 5. The P-polarized light has lower reflectivity and larger transmissivity at the surface of the imaging target 5 compared with those of the S-polarized light, and therefore, in order to obtain information of the inside of the imaging target 5, it is more effective to use the P-polarized light than to use the S-polarized light.

When a polarization plane is inclined from the P-polarized light or the S-polarized light, since reflectivity of light at a boundary of substances is different between the S-wave and the P-wave as described above, the polarization plane rotates before and after reflection. Thus, in accordance with a positional relation between three of the light source 2, the imaging target 5, and the image sensor 4, and a refractive index of the imaging target 5, the light-transmitting surface of the polarizing filter 3 may be adjusted so as to be orthogonal to the main polarization plane after reflection.

However, since such adjustment generally takes time and effort, it is desired that the special condition described above is utilized. That is, it is desired that a mode in which the light radiated from the light source 2 is approximately vertically incident on and vertically reflected by the imaging target 5 is adopted, or when the light radiated from the light source 2 is made obliquely incident on the imaging target 5, it is desired that a mode in which the light radiated from the light source 2 has a specific polarization plane such as the P-polarized light or the S-polarized light is adopted. For the light source 2 under such a condition, only by arranging the polarizing filter 3 so that the light-transmitting surface is orthogonal to the main polarization plane of the light source 2, it is possible to effectively remove mirror reflected light on the surface of the imaging target 5.

Note that, in FIG. 1, the positional relation between the three of the light source 2, the imaging target 5, and the image sensor 4 is a relation of approximately regular reflection relation with respect to light which is output in an optical axis direction of the light source 2, but the positional relation of the three is not limited thereto. Only by shifting the positional relation, even without the polarizing filter 3, it is possible to prevent light which is emitted in the optical axis direction of the light source 2 and mirror-reflected by the surface of the imaging target 5 from being directly incident on the image sensor 4.

However, laser light of a semiconductor laser is not completely liner light and is radiated with a certain constant expansion in a vertical direction and a horizontal direction. For example, some representative infrared semiconductor lasers radiate laser light having an opening angle of about 20 degrees in the vertical direction and about 10 degrees in the horizontal direction. Thus, even when the image sensor 4 is arranged at a position which is shifted from a regular reflection condition with respect to the optical axis of the light source 2, light radiated in a direction other than the optical axis direction of the light source 2 is mirror-reflected by the surface of the imaging target 5 and is incident on the image sensor 4 in some cases. Therefore, even in such a case, it is necessary that the polarizing filter 3 is laid between the imaging target 5 and the image sensor 4 so as to reliably prevent the light that is mirror-reflected by the surface of the imaging target 5 from being incident on the image sensor 4.

Note that, in Embodiment 1 to 3 described above, it is possible to use an LED (light emitting diode) that radiates light having a specific wavelength as a light source instead of the semiconductor laser. However, in this case, since light radiated from the LED is non-polarized light, when the LED is used as a light source, a polarizing filter is required to be used in order to radiate light having a specific polarization plane. Thus, it is more preferable to use a semiconductor laser.

Furthermore, one of differences between light obtained from an LED and laser light is that the laser light has a narrower wavelength width and a higher monochromaticity. Thus, it is convenient that the laser light with a high monochromaticity is used, in order to insert a narrow band-pass filter in front of the image sensor 4 and obtain biological information with a high SN ratio with respect to disturbance light that is generated from the sun, an illuminator, or the like. This also means that it is suitable that a semiconductor laser is used as the light source 2.

In order to obtain inside information of a living body, it is preferable to use an infrared ray light of which is easily absorbed by a vein. However, there is no limitation thereto in a case where the surface of the imaging target 5 is observed. For example, in a case where the S-polarized light is mirror-reflected by the surface of the imaging target 5 and thereby the surface of the imaging target 5 is observed, when visible light, a near-ultraviolet ray, or an ultraviolet ray is used in accordance with an object in addition to the infrared ray, it is possible to utilize the imaging device 1 not only for acquiring biological information of a skin surface of the imaging target 5, such as a wrinkle of the surface, a spot, a palm print, or a finger print, but also for observing a stain, a wound, contamination by a microorganism or a bacillus, or the like on the surface of the imaging target 5.

### Conclusion

An imaging device 1 according to an aspect 1 of the invention is an imaging device 1 that captures an image of a site including biological information as an imaging target 5, including: a semiconductor laser (light source 2) that radiates infrared light to the imaging target 5; a polarizing filter 3 that blocks light that is reflected by a surface of the imaging target 5 and transmits light that is reflected inside the imaging target 5; and an image sensor 4 that receives the light transmitted through the polarizing filter 3 and captures an image of the imaging target 5.

According to the aforementioned configuration, since the semiconductor laser is used as the light source 2, the light radiated from the light source 2 has a specific wavelength and a specific polarization plane. Thus, it is not necessary to use a filter such as a wavelength filter or a polarizing filter for the light source 2 according to the aspect of the invention.

Accordingly, it is possible to suppress loss of light, that is, reduction of intensity of light due to transmission through a filter. Thus, imaging accuracy of the imaging device 1 according to the aspect of invention is improved. In addition, since the filter is not required, the light source is able to be made compact and also able to be mounted on mobile equipment. Furthermore, since the filter is not required to be provided, it is possible to suppress cost of the light source 2 so as to be low.

In this manner, with the imaging device 1 according to the aspect of the invention, it is possible to improve imaging accuracy while suppressing increase in a size of the device and cost thereof.

In the imaging device 1 according to an aspect 2 of the invention, in the aspect 1, a polarizing filter 3 is arranged so that a light-transmitting surface thereof is orthogonal to a main polarization plane of the semiconductor laser.

According to the aforementioned configuration, light of the light radiated from the semiconductor laser, which is mirror-reflected by the surface of the imaging target 5, has a polarization plane which does not change, and thus the light is not transmitted through the polarizing filter 3. On the other hand, light of the light radiated from the semiconductor laser, which is diffuse-reflected inside the imaging target 5, has no specific polarization plane, and thus a part of the light is transmitted through the polarizing filter 3.

In this manner, since the polarizing filter 3 is able to block the light which is mirror-reflected by the surface of the imaging target 5, an image in which brightness distribution of the diffuse-reflected light according to the biological information is excellently reproduced is able to be captured.

In the imaging device 1 according to an aspect 3 of the invention, in the aspect 1 or 2, the semiconductor laser is an eye-safe laser.

According to the aforementioned configuration, it is possible to secure safety of a living body, particularly, an eye of a user.

In the imaging device 1 according to an aspect 4 of the invention, in the aspect 3, a polarization ratio of the semiconductor laser is not less than 3, and preferably not less than 9.

According to the aforementioned configuration, an image suitable for biometric authentication is able to be captured.

In the imaging device 1 according to an aspect 5 of the invention, in any aspect of the aspects 1 to 4, the light radiated from the semiconductor laser is P-polarized light that is obliquely incident on the imaging target 5.

When light is obliquely radiated to the imaging target 5, reflectivity of the P-polarized light at the surface of the imaging target 5 is lower compared with reflectivity of S-polarized light. Thus, according to the aforementioned configuration, since a rate of light that enters the imaging target 5 and is diffuse-reflected inside the imaging target 5 increases, and therefore imaging accuracy is able to be improved.

A biometric authentication device that includes the imaging device 1 according to any aspect of the aspects 1 to 5 described above is also included in the scope of the invention.

The imaging accuracy of the imaging device 1 according to the aspect of the invention is improved, and therefore biometric authentication accuracy of the biometric authentication device that includes the imaging device 1 is also improved.

Furthermore, a semiconductor laser for an imaging device that captures an image of a site including biological information, according to an aspect 6 of the invention, is an eye-safe laser which radiates infrared light and a polarization ratio of which is not less than 3 and preferably not less than 9.

When the semiconductor laser according to the aspect of the invention is used for the imaging device that captures an image of a site including biological information, it is possible to improve imaging accuracy while suppressing increase in a size of the device and cost thereof.

The invention is not limited to each of embodiments described above, and may be modified in various manners within the scope of the claims, and an embodiment achieved by appropriately combining technical means which are disclosed in each of different embodiments is also encompassed in the technical scope of the invention. Furthermore, by combining the technical means disclosed in each of different embodiments, a new technical feature may be formed.

### REFERENCE SIGNS LIST

1 imaging device

2 light source

3 polarizing filter

4 image sensor

5 imaging target

6 diffusion member

7 stem

8 cap

9 lead pin

10 diffusion material

12 semiconductor laser chip

13 light-emitting point

1. An imaging device that captures an image of a site including biological information as an imaging target, comprising:
a semiconductor laser that radiates infrared light, which is obtained by diffusing light radiated from a semiconductor laser chip, to the imaging target;
a polarizing filter that blocks light that is obtained by causing the infrared light to be reflected by a surface of the imaging target and transmits light that is obtained by causing the infrared light to be reflected inside the imaging target; and
an image sensor that receives the light that is transmitted through the polarizing filter and captures an image of the imaging target.

2. The imaging device according to claim 1, wherein
the polarizing filter is arranged so that a light-transmitting surface thereof is orthogonal to a main polarization plane of the semiconductor laser.

3. (canceled)

4. The imaging device according to claim 1, wherein
a polarization ratio of the semiconductor laser is not less than 3.

5. The imaging device according to claim 1, wherein
the light radiated from the semiconductor laser is P-polarized light that is obliquely incident on the imaging target.

6. A biometric authentication device comprising the imaging device according to claim 1.

7. A semiconductor laser for an imaging device that captures an image of a site including biological information, wherein
infrared light that is obtained by diffusing light radiated from a semiconductor laser chip is radiated, and
a polarization ratio is not less than 3.
